(19) United States
(12) Patent Application Publication
(10) Pub. No.: US 2024/0268759 A1
(43) Pub. Date: Aug. 15, 2024

(54) MAGNETIC-TYPE HUMAN BODY PARAMETER MEASURING DEVICE

(71) Applicant: Shenzhen Fitfaith Technology Co., Ltd., Shenzhen (CN)

(72) Inventor: Junfeng Yuan, Shenzhen (CN)

(21) Appl. No.: 18/451,397

(22) Filed: Aug. 17, 2023

(30) Foreign Application Priority Data
Feb. 9, 2023 (CN) 2023101382515
Feb. 9, 2023 (CN) 2023202436378

(51) Int. Cl. A61B 5/00

(52) U.S. Cl. CPC A61B 5/683; A61B 5/742; A61B 2562/0247; A61B 2562/0271; A61B 2562/14; A61B 2562/166

(57) ABSTRACT

The embodiment of the invention provides a magnetic-type human body parameter measuring device, comprising the first circuit assembly arranged in the main body and the first circuit assembly is electrically connected with the first sensor; a main body and magnetic bandages used to form an annular shape to bind the limb in a magnetic mode, and the first sensor faces the limb; the annular shape that the main body and the magnetic bandage formed is equipped with first adaptive cushion in it. The measurement to human body parameter is realized through first sensor; when wearing, the annular size to bind the limb is adjusted in order to adapt to different limbs through the magnetic bandage with magnetic design, such as the size of finger, and annular size can be further adjusted through first adaptive cushion; on the one hand, size is adjusted more conveniently to make annular more fit the wearing position; on the other hand, the pressure to the wearing position has been alleviated and comfort level of wearing is raised, thereby improving user experience.

### 1. TECHNICAL FIELD

The invention relates to the technical field of medical device, specifically relating to a magnetic-type human body parameter measuring device.

### 2. BACKGROUND ART

The four vital signs include human parameters of temperature, pulse, respiration and blood pressure, and they constitute the pillar of maintaining normal body activities, none of which is dispensable; abnormality of any above sign can lead to severe or fatal disease, and at the same time, some diseases can bring about changes or deterioration of these four vital signs; therefore, monitoring blood oxygen saturation, respiratory rate, pulse rate and other physiological parameters is of great significance to know about human health status.

It's of vital importance to constantly measuring human's physiological parameters such as blood oxygen saturation, pulse rate, respiratory rate, and temperature. At present, the common fixing methods for human parameter measuring device (such as blood oxygen sensor) is at fingers, toes, soles of feet, ears, etc. mainly utilize elasticity of metal or silica gel, and give the measuring position a clamping force to fix, the size of which is difficult to adapt to the measuring positions with different perimeters and sizes.

Most of the wearing methods at fingers and toes adopt clamps, which exerts a relative large pressure on the measuring position, the detected person is tend to feel not quite comfortable, so the measuring device is not suitable for wearing for a long time, particularly, when monitoring during sleep, it will cause difficulty falling asleep, light sleep, and easy to wake up at midnight or the clamps easily fall off, affecting sleeping speed and the person is not easy to fall into deep sleep in the sleeping process, so the influence on sleep stages is large and wearing for long time is prone to damage human tissues of the measured part.

### 3. SUMMARY OF THE INVENTION

Given above mentioned problems, the invention provides a magnetic-type human body parameter measuring device to overcome above problems or, at least, partial problems. The invention aims to solve problems of excessive pressure on the measuring position and lack of comfortable.

A magnetic-type human body parameter measuring device comprising a main body and magnetic bandages;

The first circuit assembly is arranged in the main body and said first circuit assembly is electrically connected with the first sensor; and/or the second circuit assembly is provided in said magnetic bandages and said second circuit assembly is electrically connected with the second sensor;

Said main body and magnetic bandages are used to form an annular shape to bind the limb in a magnetic mode, and said first sensor and/or second sensor face the limb;

Said annular shape that the main body and the magnetic bandages formed is equipped with first adaptive cushion therein.

Preferably, said magnetic bandages include an inner bandage and an outer bandage;

Said inner bandage and outer bandage are respectively connected on the one end of said main body and a plurality of magnets are arranged in the bandages; wherein said inner bandage and outer bandage are attracted to each other and form an annular shape via the main body to bind the limb.

Preferably, said inner bandage includes a groove, a FPC board used to bear said second circuit assembly and a covering layer;

Said FPC board is arranged inside said groove, and said covering layer is matched with the shape of said groove and covers said FPC board;

Said second sensor is arranged on one end of said FPC board and its surface is exposed to the adaptive hole provided on said covering layer.

Preferably, the surface of said magnetic bandages is made of elastic material; said elastic material includes silica gel, rubber, TPU, and TPE material.

Preferably, in the middle of said first adaptive cushion is provided with the first through-hole used to detect signal via the sensor.

Preferably, said first adaptive cushion includes little finger circumference adaptive cushion, middle finger circumference adaptive cushion, and thumb circumference adaptive cushion.

Preferably, it also includes a second adaptive cushion wherein said second adaptive cushion is provided with the second through-hole used to detect signal via the sensor.

Preferably, a display screen is arranged on the surface of said main body, and said display screen is electrically connected with said first circuit assembly.

Preferably, the surface of said outer bandage and inner bandage is provided with a vent groove.

Preferably, said first sensor includes a light receiving tube, a light-emitting tube, a temperature sensor, a pressure sensor and a motion sensor.

Preferably, said second sensor includes a light receiving tube, a light-emitting tube, a temperature sensor or a pressure sensor.

Preferably, said magnets are N52 strong magnets.

Preferably, said first circuit assembly includes a wireless data transmission component, a vibration component, a sound component, a USB interface, a charging port and a contact.

Preferably, said second circuit assembly includes a wireless data transmission component, a vibration component, a sound component, a USB interface, a charging port and a contact.

Preferably, it also includes a third adaptive cushion; said third adaptive cushion is arranged within said annular shape formed by said main body and magnetic bandages when in use;

Said third adaptive cushion includes the first silica gel layer, the second silica gel layer and the third magnet; wherein said first silica gel layer is provided with the third through-hole corresponding to said first sensor in position; said first silica gel layer and the second silica gel layer are buckled to form a cushion space; said third magnet is fixedly arranged in said cushion space.

The embodiment of the invention has following advantages:
The embodiment of the invention comprises a main body and magnetic bandages; the first circuit assembly is arranged in the main body and said first circuit assembly is electrically connected with the first sensor; and/or the second circuit assembly is provided in said magnetic bandages and said second circuit assembly is electrically connected with the second sensor; said main body and magnetic bandages are used to form an annular shape to bind the limb in a magnetic mode, and said first sensor and/or second sensor face the limb; the annular shape that the main body and the magnetic bandages formed is equipped with first adaptive cushion therein. The measurement to human body parameter is realized through first sensor and/or second sensor; when wearing, the annular size to bind the limb is adjusted in order to adapt to different limbs through the magnetic bandages with magnetic design, such as the size of finger, and annular size can be further adjusted through first adaptive cushion; on the one hand, size is adjusted more conveniently to make annular more fit the wearing position; on the other hand, the pressure to the wearing position has been alleviated and comfort level of wearing is raised, thereby improving user experience.

### 4. BRIEF DESCRIPTION OF ACCOMPANY DRAWINGS

In order to illustrate the technical scheme of the invention more clearly, drawings needed for the illustration of the invention is further described in brief. Obviously, drawings to be described hereinafter are only partial embodiments of the invention. It is to be understood by those skilled in the art that other drawings can be obtained based on the drawings of the invention with non-creative labor.

FIG. 1 is the structural diagram of a magnetic-type human body parameter measuring device provided by an embodiment in the invention;

FIG. 2 is the structural diagram of the first adaptive cushion of a magnetic-type human body parameter measuring device provided by an embodiment in the invention;

FIG. 3 is the structural diagram of the second adaptive cushion of a magnetic-type human body parameter measuring device provided by an embodiment in the invention;

FIG. 4 is the structural diagram of the magnetic-type human body parameter measuring device arranged with a plurality of first adaptive cushions provided by an embodiment in the invention;

FIG. 5 is the the structural diagram of the magnetic-type human body parameter measuring device arranged with the first adaptive cushion and second adaptive cushion provided by an embodiment in the invention;

FIG. 6 is the structural diagram of the inner bandage of a magnetic-type human body parameter measuring device provided by an embodiment in the invention;

FIG. 7 is the structural diagram of the outer bandage of a magnetic-type human body parameter measuring device provided by an embodiment in the invention;

FIG. 8 is the structural diagram of the third adaptive cushion of a magnetic-type human body parameter measuring device provided by an embodiment in the invention;

As shown in the accompanying drawings: 1 main body, 2 magnetic bandage, 3 first adaptive cushion, 4 second adaptive cushion, 5 third adaptive cushion, 11 first sensor, 12 display screen, 20 vent groove, 21 outer bandage, 22 inner bandage 23 second sensor, 24 covering layer, 25 FPC board, 26 groove, 27 first magnet, 28 adaptive hole, 29 second magnet, 31 first through-hole, 41 second through-hole, 51 first silica gel layer, 52 second silica gel layer, 53 third magnet, 54 third through-hole

### 5. SPECIFIC EMBODIMENT OF THE INVENTION

In order to illustrate the purpose, technical scheme and advantages of the embodiments of the invention more clearly, the technical scheme of the preferred embodiments is further described clearly and completely. Obviously, only partial embodiments of the invention are shown and the actual structure is not limited thereto. All other embodiments, which can be obtained by those skilled in the art without making any creative effort based on the embodiments in the present invention, shall all fall within the protective scope of the invention.

It should be noted that, in any embodiment of the present application, the FPC board (Flexible Printed Circuit) is a Flexible Printed Circuit board which is made of polyimide or polyester film as a base material and has high reliability and excellent properties, and has the characteristics of high wiring density, light weight, thin thickness and good bending property; USB (Universal Serial Bus) is an external Bus standard for standardizing connection and communication between computer and external devices, and is an interface technology applied in the field of PC. The TPU is a thermoplastic polyurethane elastomer (TPU), and is an elastomer which can be plasticized by heating and can be dissolved by a solvent, and has excellent comprehensive properties of high strength, high toughness, wear resistance, oil resistance, etc. TPE (Thermoplastic elastomer), TPE or TPR for short, is a kind of elastomer having rubber's elasticity at normal temperature and plastic molding at high temperature

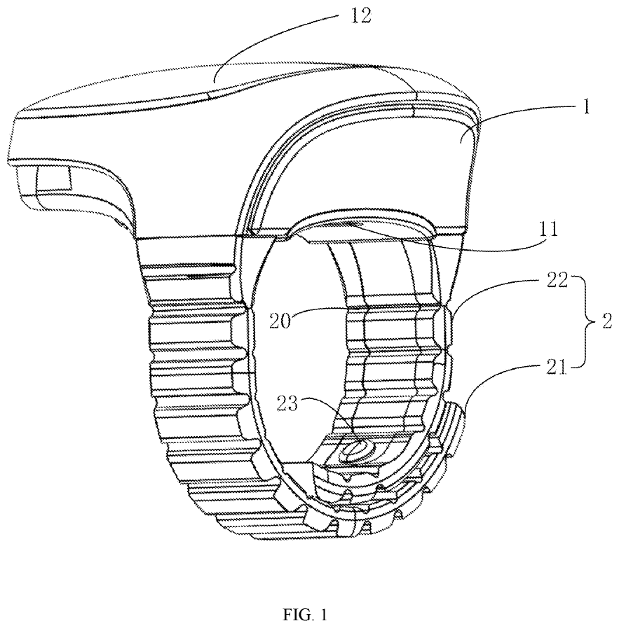

With reference to FIG. 1, it illustrates the structural diagram of a magnetic-type human body parameter measuring device, comprising a main body 1 and magnetic bandages 2; the first circuit assembly is arranged in the main body 1 and said first circuit assembly is electrically connected with the first sensor 11; and/or the second circuit assembly is provided in said magnetic bandages 2 and said second circuit assembly is electrically connected with the second sensor 23; said main body 1 and magnetic bandages 2 are used to form an annular shape to bind the limb in a magnetic mode, and said first sensor and/or second sensor 23 face the limb; said annular shape that the main body 1 and the magnetic bandages 2 formed is equipped with first adaptive cushion 3 therein.

In above-mentioned embodiment, the measurement to human body parameter is realized through first sensor 11 and/or second sensor 23; when wearing, the annular size to bind the limb is adjusted in order to adapt to different limbs through the magnetic bandages 2 with magnetic design, such as the size of finger, and annular size can be further adjusted through first adaptive cushion 3; on the one hand, size is adjusted more conveniently to make annular more fit the wearing position; on the other hand, the pressure to the wearing position has been alleviated and comfort level of wearing is raised, thereby improving user experience.

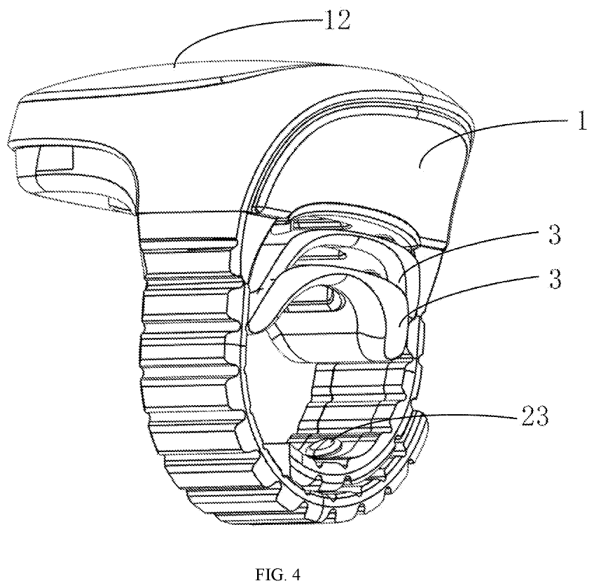

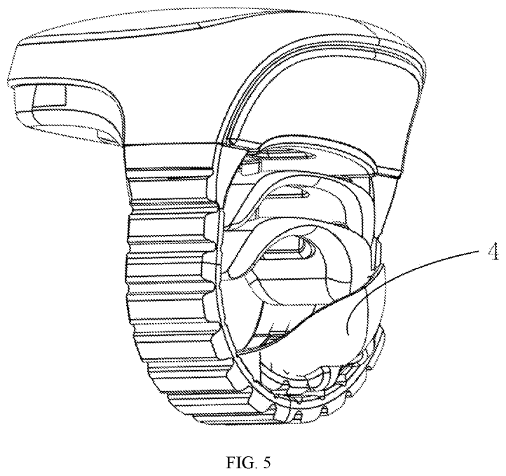

In an embodiment of the invention, as is shown in FIG. 1, FIG. 4 and FIG. 5, said magnetic bandages 2 includes an inner bandage 22 and an outer bandage 21; said inner bandage 22 and outer bandage 21 are respectively connected on the one end of said main body 1 and a plurality of magnets are arranged in the bandages; wherein said inner bandage 22 and outer bandage 21 are attracted to each other and form an annular shape via the main body 1 to bind the limb.

As an embodiment, aimed at the defects of prior body parameter measuring device, the invention provides a body parameter measuring device, comprising a measuring device main body 1 and two magnetic bandages 2; According to the positions of wearing, the bandages are divided into the inner bandage 22 and the outer bandage 21, wherein the inner bandage 22 and outer bandage 21 can be arranged with sensor unit or other electronic components; the main body 1 and magnetic bandages 2 utilize magnetism to constitute an annular shape and placed on the finger, the perimeter of the annular shape can be adjusted to adapt to different finger sizes.

In a specific implementation, when the human body parameter measuring device is placed on the fingers, toes or the soles of the feet of the neonate, the main body 1 of the device, the inner bandage 22 and the outer bandage 21, the first sensor 11 and the second sensor 23 (which are simply called sensor combination) contained therein comprise at least one luminous tube and one receiving tube to form a loop, the photoelectric plethysmogram signals of the fingers, the toes or the soles of the feet of the neonate and the like are measured, and the signal is used for further calculating vital signs of the human body such as blood oxygen saturation, pulse rate, respiratory rate, perfusion index and the like, wherein, the sensor combination can also comprise a plurality of (more than one) luminous tubes and a plurality of receiving tubes, the luminous tubes respectively emit light pulses in a time-sharing way, the photoelectric plethysmogram signals received by each receiving tube are judged, and the luminous tube-receiving tube combination with better signal quality is preferentially selected for display and calculation.

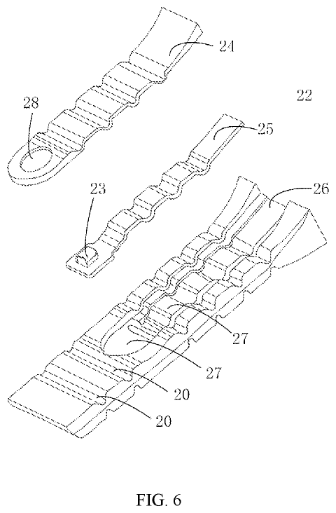

In an embodiment of the invention, as is shown in FIG. 6, said inner bandage 22 includes a groove 26, a FPC board 25 used to bear said second circuit assembly and a covering layer 24; said FPC board 25 is arranged inside said groove 26, and said covering layer 24 is matched with the shape of said groove 26 and covers said FPC board 25; said second sensor 23 is arranged on one end of said FPC board 25 and its surface is exposed to the adaptive hole 28 provided on said covering layer 24.

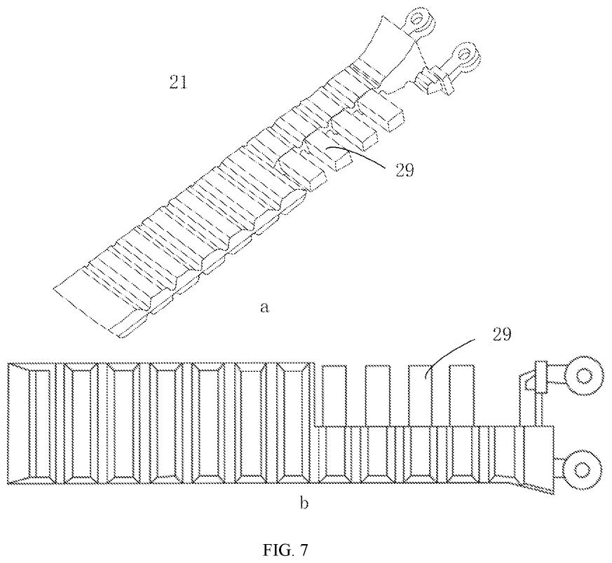

In a specific implementation, said inner bandage 22 comprises a covering layer 24, a FPC board (Flexible Printed Circuit) 25, the second sensor 23, and the first magnet 27, wherein the FPC board fits into the inside of the groove 26 of the inner bandage 22, the covering layer 24 and the inner bandage 22 assemble the the FPC board, controllers needed, and the second sensor 23 (sensor module) inside the magnetic inner bandage 22, realizing inserting sensor units into the magnetic inner bandage 2; wherein, with reference to FIG. 7, the second magnet 29 is provided in said outer bandage 21, said first magnet 27 and second magnet 29 are respectively wrapped in said inner bandage 22 and outer bandage 21 to make them attract each other. Preferably, a plurality of sensor units can be arranged on the FPC board, including but not limited to the light-emitting component, light receiving sensor, controller, etc. A plurality of sensor units can also be arranged on the magnetic outer bandage 21, including but not limited to the light-emitting component, light receiving sensor, controller, etc.

In an embodiment of the invention, with the aim of further enhancing the comfort level of wearing, the surface of said magnetic bandage 2 is made of elastic material; said elastic material includes silica gel, rubber, TPU, and TPE material.

In an embodiment, said magnetic bandage 2 is made of elastic material such as silica gel, rubber, TPU, and TPE material. The magnet is encased within the elastic material, wherein both the inner bandage 22 and the outer bandage 21 may leave room for wires and one or more second sensors 23. Said second sensor 23 can be a light receiving tube, a light-emitting tube, a temperature sensor or a pressure sensor, which can also be other sensor components. The interior main body 1 of the measuring device are arranged with the circuit (first circuit assembly), and both the inner bandage 22 and the outer bandage 21 can be placed with one or more second sensors 23, or can be placed with nothing.

It should be noted that, said first sensor 11 and second sensor 23 can be identical sensor unit, or different sensor units, or mutually independent sensor units, or mutually-coordinate sensor units; for instance, one of them as signal sending unit while the other one as signal receiving unit, and many other combinations.

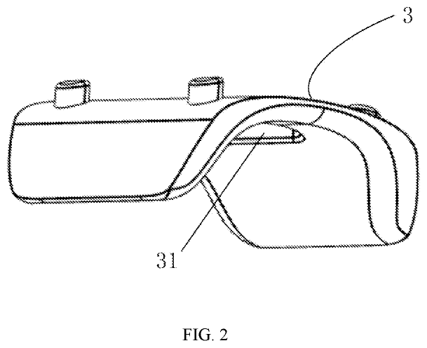

In an embodiment of the invention, with reference to FIG. 2, in the middle of said first adaptive cushion 3 is provided with the first through-hole 31 used to detect signal via the sensor.

In above embodiment, said first adaptive cushion 3 improved the comfort level of wearing and enables the annular shape for wearing formed by the device has more suitable size and tightness; the sensor detects signal through the first through-hole 31 enjoys better signal channel.

Furthermore, said first adaptive cushion 3 includes little finger circumference adaptive cushion, middle finger circumference adaptive cushion, and thumb circumference adaptive cushion. The first adaptive cushions 3 with different types enable it be wore on various fingers or toes, thus having excellent adaptability.

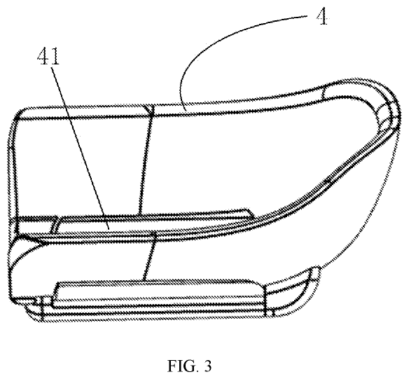

In an embodiment of the invention, with reference to FIG. 3, it also includes a second adaptive cushion 4 wherein said second adaptive cushion 4 is provided with the second through-hole 41 used to detect signal via the sensor.

In an embodiment, with reference to FIG. 5, said body parameter measuring device can also be matched with two types of cushions, namely, said first adaptive cushion 3 and second adaptive cushion 4, after being mounted on said main body 1 and said magnetic bandages 2 the device can better fit different fingers or toes, which can not only realize better wearing comfort level, but also obtain better measuring result. The first adaptive cushion is arranged at the lower part of the main machine and said second adaptive cushion 4 is arranged on the inner bandage; there are a plurality of approaches to fit the finger, such as the following four typical combinations: a. only the first adaptive cushion 3 is used to adapt the fingers; b. only using a cushion mounted or integral with the inner bandage to fit the fingers; c. wrapping the fingers with the first adaptive cushion 3 and second adaptive cushion 4 together; d. no cushion is used. The adaptive cushion can be dismantled, washed, disinfected, and removed, and every kind of cushions provides the specification of multiple sizes; the cushion can transform the power of constraining force of bandage into the upper and lower clamp force to the finger and improves measurement accuracy and comfort level.

It should be noted that, with reference to FIG. 4, in order to better fit the finger, said main body 1 can be arranged with a plurality of first adaptive cushions 3.

In an embodiment of the invention, with reference to FIG. 1 and FIG. 4, a display screen 12 is arranged on the surface of said main body 1, and said display screen 12 is electrically connected with said first circuit assembly.

Preferably, said first circuit assembly includes a wireless data transmission component, a vibration component, a sound component, a USB interface, a charging port and a contact.

In an embodiment of the invention, with reference to FIG. 6 and FIG. 7, the surface of said outer bandage 21 and inner bandage 22 is provided with a vent groove 20.

In a specific embodiment, said magnetic bandage 2 is integrally-formed by elastic material with placing cavities put in order therein, wherein each said placing cavity is provided with magnetic component, and when two magnetic bandages 2 are provided, the magnetic action enables a pair of magnetic bandages 2 attracted to each other so as to adjust the fixation of a pair of magnetic bandages 2; and a plurality of magnetic components can also add the firmness of magnetic bandages 2 after being magnetically attracted and realize diversity of adjustment; hollow vent grooves 20 are provided on adjacent two placing cavities; when a plurality of placing cavities are arranged, the vent grooves 20 are also put in order, which enables said magnetic bandage 2 can be applied to the skin and vent grooves 20 make gap exists between the magnetic bandage 2 and the skin, avoiding the magnetic bandage 2 attached to the skin too tight when in usage, further improving users' comfortable feeling.

It should be noted that said magnetic component mainly adopt magnets, specifically, said magnets are preferred to be N52 strong magnets.

In an embodiment of the invention, said first sensor 11 includes a light receiving tube, a light-emitting tube, a temperature sensor, a pressure sensor and a motion sensor. Said second sensor 23 includes a light receiving tube, a light-emitting tube, a temperature sensor or a pressure sensor.

In an embodiment of the invention, said second circuit assembly includes a wireless data transmission component, a vibration component, a sound component, a USB interface, a charging port and a contact.

In a specific implementation, the second sensor 23 in said magnetic bandages 2 can also be the temperature sensor, which also includes a heating element and a controller; the output end of said temperature sensor is connected with the input end of said controller and the output end of said controller is connected with the heating element; the temperature data measured by said temperature sensor will be transmitted to said controller, and said controller will judge whether to switching on or off said heating element according to the temperature data of the structure, wherein critical temperature value is set in the controller; when the temperature data received is less than the critical temperature value, the controller switches on the heating element, which begins to heat the finger to be measured to obtain better measuring effect; when the temperature data received is greater than the critical temperature value, the controller will default to not switching on the heating element; of course, the magnetic bandages 2 can be arranged with a cooling element, and when the temperature data received is greater than the critical temperature value, the controller will switch on the cooling element for cooling.

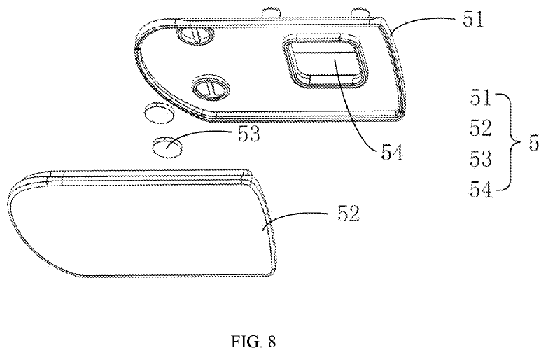

In an embodiment of the invention, with reference to FIG. 8, it also includes a third adaptive cushion 5; said third adaptive cushion 5 is arranged within said annular shape formed by said main body 1 and magnetic bandage when in use; said third adaptive cushion 5 includes the first silica gel layer 51, the second silica gel layer 52 and the third magnet 53; wherein said first silica gel layer 51 is provided with the third through-hole 54 corresponding to said first sensor in position; said first silica gel layer 51 and the second silica gel layer 52 are buckled to form a cushion space; said third magnet 53 is fixedly arranged in said cushion space. When in use, the third through hole 54 is mounted corresponding to said sensor 11. As the cushion space is provided with the third magnet 53, which is preferred to be N52 strong magnets and enable the first silica gel layer 51 to fits into the bottom surface of the main body 1 under the magnetic action; as there is air in the cushion space, wearing for long time can make the finger more comfortable.

It should be noted that, in order to simplify the description, all embodiments are illustrated in a series of actions, but it should be understood by those skilled in the art that the embodiments are not limited to the the action sequence of description. Some steps can be implemented in other sequences or simultaneously. It should also be understood by those skilled in the art that all the embodiments described in the invention are preferred embodiments, actions involved are not mandatory for the embodiment of the invention.

The embodiments in the present specification are all described in a progressive manner, and each embodiment focuses on differences from other embodiments, and portions that are the same and similar between the embodiments may be referred to each other.

As will be understood by those skilled in the art, embodiments of the present invention may be provided as a method, apparatus, or computer program product. Accordingly, embodiments of the present invention may take the form of an entirely hardware embodiment, an entirely software embodiment or an embodiment combining software and hardware aspects. Furthermore, embodiments of the present invention may take the form of a computer program product embodied on one or more computer-usable storage media (including, but not limited to, disk storage, CD-ROM, optical storage, etc.) having computer-usable program code embodied therein.

Embodiments of the present invention are described with reference to flowchart illustrations and/or block diagrams of methods, terminal devices (systems), and computer program products according to embodiments of the invention. It will be understood that each flow and/or block of the flowchart illustrations and/or block diagrams, and combinations of flows and/or blocks in the flowchart illustrations and/or block diagrams, can be implemented by computer program instructions. These computer program instructions may be provided to a processor of a general purpose computer, special purpose computer, embedded processor, or other programmable data processing terminal to produce a machine, such that the instructions, which execute via the processor of the computer or other programmable data processing terminal, create means for implementing the functions specified in the flowchart flow or flows and/or block diagram block or blocks.

These computer program instructions may also be stored in a computer-readable memory that can direct a computer or other programmable data processing apparatus to function in a particular manner, so that the instructions stored in the computer-readable memory produce an article of manufacture including instruction means which implement the function specified in the flowchart flow or flows and/or block diagram block or blocks.

These computer program instructions may also be loaded onto a computer or other programmable data processing terminal to cause a series of operational steps to be performed on the computer or other programmable terminal to produce a computer implemented process so that the instructions which execute on the computer or other programmable terminal to provide steps for implementing the functions specified in the flowchart flow or flows and/or block diagram block or blocks.

Although preferred embodiments of the present invention have been described, those skilled in the art may make additional changes and modifications to these embodiments once the basic inventive concepts are known. Therefore, the appended claims are intended to be construed to include the preferred embodiments and all changes and modifications that fall within the scope of the embodiments of the invention.

Finally, it should also be noted that, in this invention, relational terms such as first and second, and the like are used solely to distinguish one entity or action from another entity or action without necessarily requiring or implying any actual such relationship or order between such entities or actions. Also, the terms “include”, “comprise” or any other variations thereof are intended to cover non-exclusive inclusion, accordingly a process, method, article, or terminal device including a series of elements includes not only those elements but also other elements not explicitly listed, or further includes elements inherent to such process, method, article, or terminal device. More restrictively, the elements defined by the statement “including a . . . ” do not exclude the existence of additional identical elements in the process, method, article or terminal device including said elements.

The magnetic-type human body parameter measuring device provided by the invention are introduced in detail and specific embodiments are applied to illustrate the principle and implementation approach of the invention. The above description is only for helping to understand the method of the invention and its core idea; at the same time, for the general technical person in the field, according to the idea of the invention, there will be changes in the specific implementation and application scope. In summary, the content of this specification should not be understood as a limitation of the invention.

1. A magnetic-type human body parameter measuring device comprising a main body and magnetic bandages;
the first circuit assembly is arranged in the main body and said first circuit assembly is electrically connected with the first sensor; and/or the second circuit assembly is provided in said magnetic bandages and said second circuit assembly is electrically connected with the second sensor;
said main body and magnetic bandages are used to form an annular shape to bind the limb in a magnetic mode, and said first sensor and/or second sensor face the limb;
said annular shape that the main body and the magnetic bandages formed is equipped with first adaptive cushion therein.

2. The magnetic-type human body parameter measuring device of claim 1 wherein said magnetic bandages include an inner bandage and an outer bandage;
said inner bandage and outer bandage are respectively connected on the one end of said main body and a plurality of magnets are arranged in the bandages; wherein said inner bandage and outer bandage are attracted to each other and form an annular shape via the main body to bind the limb.

3. The magnetic-type human body parameter measuring device of claim 2 wherein said inner bandage includes a groove, a FPC board used to bear said second circuit assembly and a covering layer;
said FPC board is arranged inside said groove, and said covering layer is matched with the shape of said groove and covers said FPC board;
said second sensor is arranged on one end of said FPC board and its surface is exposed to the adaptive hole provided on said covering layer.

4. The magnetic-type human body parameter measuring device of claim 2 wherein the surface of said magnetic bandages is made of elastic material; said elastic material includes silica gel, rubber, TPU, and TPE material.

5. The magnetic-type human body parameter measuring device of claim 1 wherein in the middle of said first adaptive cushion is provided with the first through-hole used to detect signal via the sensor.

6. The magnetic-type human body parameter measuring device of claim 5 wherein said first adaptive cushion includes little finger circumference adaptive cushion, middle finger circumference adaptive cushion, and thumb circumference adaptive cushion.

7. The magnetic-type human body parameter measuring device of claim 1 wherein it also includes a second adaptive cushion wherein said second adaptive cushion is provided with the second through-hole used to detect signal via the sensor.

8. The magnetic-type human body parameter measuring device of claim 1 wherein a display screen is arranged on the surface of said main body, and said display screen is electrically connected with said first circuit assembly.

9. The magnetic-type human body parameter measuring device of claim 2 wherein the surface of said outer bandage and inner bandage is provided with a vent groove.

10. The magnetic-type human body parameter measuring device of claim 1 wherein said first sensor includes a light receiving tube, a light-emitting tube, a temperature sensor, a pressure sensor and a motion sensor.

11. The magnetic-type human body parameter measuring device of claim 1 wherein it also includes a third adaptive cushion; said third adaptive cushion is arranged within said annular shape formed by said main body and magnetic bandages when in use;
said third adaptive cushion includes the first silica gel layer, the second silica gel layer and the third magnet; wherein said first silica gel layer is provided with the third through-hole corresponding to said first sensor in position; said first silica gel layer and the second silica gel layer are buckled to form a cushion space; said third magnet is fixedly arranged in said cushion space.
